(19) United States
(12) Patent Application Publication
(10) Pub. No.: US 2026/0325782 A1
(43) Pub. Date: Oct. 22, 2026

(54) NO DELIVERY APPARATUS WITH AMBIENT-AIR SAMPLING LINE

(71) Applicant: INOSYSTEMS, ANTONY (FR)

(72) Inventors: Nathan PROUVEZ, ANTONY (FR); Fernando REY MARTIN, ANTONY (FR); Axelle SAAS, ANTONY (FR); Nicolas NOTIS, ANTONY (FR)

(21) Appl. No.: 19/544,611

(22) Filed: Feb. 19, 2026

(30) Foreign Application Priority Data
Apr. 18, 2025 (FR) 2504173

(51) Int. Cl. A61M 16/08; A61M 16/10; A61M 16/20

(52) U.S. Cl. CPC A61M 16/085; A61M 16/104; A61M 16/202; A61M 2202/0275; A61M 2205/505

(57) ABSTRACT

The invention relates to an NO delivery apparatus (1) before carrying a gaseous NO/nitrogen mixture and injecting it into a patient circuit (2); a main gas sampling line (4); a gas analysis line (5) measuring the NO and/or NO₂ contents; gas suction means (7); operating means (8); and display means (9) for displaying the NO and/or NO₂ content. A secondary gas sampling line (20) communicates with the atmosphere. Valve means (21) coupled to the main line (4), to the secondary line (20) and to the analysis line (5) are operated by the operating means (8) in order to place the gas analysis line (5) in communication with one or the other of the gas sampling lines (4, 20). Gas administration installation including the apparatus (1).

### CROSS-REFERENCE TO RELATED APPLICATIONS

This patent application claims priority to French Patent Application No. 2504173, filed Apr. 18, 2025 and the entire contents of which are incorporated herein by reference.

### TECHNICAL FIELD

The invention relates to an NO delivery device or apparatus, namely to an apparatus for supplying an NO-containing gas, which is intended to be connected to one or more pressurized gaseous NO source(s), such as (an) NO/N₂ cylinder(s), and the patient circuit supplied by a medical ventilator, said NO delivery apparatus comprising gas analysis means making it possible to measure the NO and/or NO₂ content in the final gas flow supplied to the patient or, as the case may be, in the ambient air, which is to say the air of the location in which the NO delivery apparatus is situated, such as a room, a ward, a vehicle or the like, and display means for displaying the corresponding NO and/or NO₂ values; and to an installation for administering gas to a patient and comprising such an NO delivery apparatus.

### BACKGROUND

Nitric oxide, or NO, is commonly used to treat various medical conditions, as described in particular by EP-A-560928, EP-A-1516639 and U.S. Pat. No. 10,201,564. In general, a gaseous mixture of NO (i.e. a few ppm by vol.) and of nitrogen (N₂) is injected by an NO delivery device into an oxygen-containing gaseous flow such as an N₂/O₂ mixture or air, or even pure oxygen, which is carried by the patient circuit from a gas supply installation such as a medical ventilator which supplies the oxygen-based gaseous flow to said patient circuit, and the final gaseous (NO/N₂/O₂) mixture thus obtained, containing NO, oxygen and nitrogen, is then administered by inhalation to the patient to be treated having need thereof, either invasively using a tracheal tube, or non-invasively using a mask or the like, as described in U.S. Pat. No. 5,558,083.

In order to ensure that the final gaseous NO/N₂/O₂ flow contains no, or only a negligible quantity of, toxic species, i.e. NO₂ compounds resulting from unwanted oxidation of some of the NO by the O₂, the NO delivery apparatus has a gas sampling line making it possible to take a small quantity, i.e. a sample, of the final gaseous (NO/N₂/O₂) mixture within the respiratory circuit, as described in US2006/207594 or EP4537879. These gas samples are then analysed in an analysis line of the NO delivery apparatus to verify whether the composition of the final gaseous NO/N₂/O₂ flow corresponds to that desired, notably its NO content which has to be below 80 ppmv, and especially its NO₂ content which has to be as low as possible, typically under 5 ppmv because NO₂ is a gas that is toxic and an irritant and can produce serious lesions in the patient, even at low concentration.

The problem is that the medical staff, i.e. the doctors, nurses or the like, or allied health professionals, may be exposed in various ways to these potentially harmful and irritant gases, i.e. NO and NO₂, within hospital establishments or in transport vehicles because these gases are found in the ambient air of the enclosed spaces in which NO treatment installations are used, such as hospital wards, operating theatres or resuscitation rooms, corridors or other types of room, and also in the passenger spaces of transport vehicles, particularly emergency-response vehicles, such as ambulances, helicopters, aircraft, etc.

Under various circumstances, these compounds, NO and NO₂, accumulate in the ambient air around the NO supply installation and the caregiving staff may find themselves inhaling them, inadvertently and/or unavoidably, which is to say unintentionally.

Thus, when the flexible hoses that connect the NO/N₂ cylinders to the NO delivery device are being purged, typically when an NO cylinder is being replaced, or when the device is being started, some of the gas, i.e. of the NO/N₂ mixture, contained in the flexible hose or conduit is discharged via a discharge tube and therefore finds its way into the hospital room or the passenger space of a transport vehicle.

Likewise, the gases exhaled by the patient also contain residual NO which is discharged into the atmosphere via the expiratory valve of the ventilator.

Moreover, and similarly, NO may also be found in the ambient atmosphere around the patient if the ventilator or the patient circuit becomes inadvertently disconnected, and/or in the case of non-invasive ventilation (NIV) by mask or high flow nasal cannula ventilation.

In addition, some of the NO found in the atmosphere of the hospital room or in the passenger space of a transport vehicle, inevitably oxidizes to NO₂ upon contact with the oxygen present in the air.

Because NO compounds, and especially NO₂, are toxic to the medical or allied health professionals, maximum exposure limits have been set by the Health Authorities. Thus, in France, the occupational exposure limit (OEL) is set at 2 ppm for NO and at 0.5 ppm for NO₂ for an 8-hour exposure, and at 1 ppm for a short-term exposure of, for example, 15 minutes.

One problem is that certain caregivers, chiefly the allied health professionals, are afraid of being exposed to significant amounts of NO and of NO₂ when caring for patients being treated by the administration of NO, causing them anxiety and moreover potentially affecting their health, particularly if the maximum exposure limits are exceeded.

Personal protective equipment for monitoring NO and NO₂ does exist, but this is chiefly intended for use in industry because it is unsuitable for, or ill-suited to, use in a hospital and/or in the passenger space of a transport vehicle, i.e. equipment is not always available and/or functional, care teams work on rotation, caregivers may not be aware of its existence and/or of where it is stored, complex to use in emergency-response vehicles, etc.

NO apparatus fitted with a gas analysis line intended for example for calibrating the apparatus is also known, for example from EP4316555, EP4537879, US2017/074845, EP4434565 and EP2522384.

From that starting point, one problem is that of being able to determine the concentration of potentially toxic compounds, particularly the compounds NO and/or NO₂, in the ambient air of a hospital room or of the passenger space of a transport vehicle in which an NO delivery apparatus and/or (an) NO supply installation(s) is (are) situated and/or being used, for example a ward, a resuscitation room, the passenger space of an ambulance, of a helicopter, of an aircraft or the like, so as to reduce the anxiety of the caregivers and especially preserve their health by allowing them to assure themselves that they are not being subjected to excessive toxic doses typically exceeding the OEL or the STEL (short-term exposure limit).

### SUMMARY

A solution according to the invention concerns an NO delivery device or apparatus, namely an apparatus for supplying an NO-containing gas, typically an NO/nitrogen mixture, comprising:
a main gas circuit for carrying an NO-containing gas, typically an NO/N₂ mixture, as far as at least one gas outlet configured to be fluidically coupled to a patient circuit carrying an oxygen-containing respiratory gas, such as air or an oxygen/nitrogen mixture,
a main gas sampling line configured to be fluidically coupled to said patient circuit,
a gas analysis line comprising concentration-measuring means configured to perform NO and/or NO₂ content measurements within the gaseous flow present in said gas analysis line,
gas suction means configured to suck the gas into at least the gas analysis line and cause it to circulate therein,
microprocessor-containing operating means configured to process the concentration measurements taken by the concentration-measuring means and from them deduce at least an NO and/or an NO₂ content, and
display means configured to display said at least one NO and/or NO₂ content.

In addition, according to the invention, the apparatus further comprises a secondary gas sampling line communicating fluidically with the ambient atmosphere, i.e. the atmospheric air, and valve means fluidically coupled to the main gas sampling line, to the secondary gas sampling line and to the gas analysis line, and the operating means are further configured to control the valve means in order to place the gas analysis line in fluidic communication with one or, alternatively, with the other, of the main gas sampling line and secondary gas sampling line.

Depending on the embodiment considered, the apparatus of the invention can comprise one or more of the following features:
the valve means comprise an electrically-operated valve comprising: a main valve inlet fluidically coupled to the main gas sampling line, a secondary valve inlet fluidically coupled to the secondary gas sampling line, and a valve outlet fluidically coupled to the gas analysis line.
the operating means are configured to control the valve means, particularly the electrically-operated valve, in order to place the gas analysis line in fluidic communication with (i.e. switch it between):
   either the main gas sampling line in order to supply the gas analysis line with gas coming from the patient circuit, namely typically a gaseous mixture containing NO, nitrogen and oxygen,
   or the secondary gas sampling line in order to supply the gas analysis line with ambient air coming from the atmosphere surrounding the apparatus 1, namely the atmospheric air present at the location at which the apparatus is situated.
the valve means comprise an electrically-operated valve.
the electrically-operated valve is operated, i.e. controlled or commanded, by the operating means.
the electrically-operated valve is of the 3-way type, its three ports being the 2 valve inlets and the valve outlet.
the electrically-operated valve is electrically connected to the operating means.
according to another embodiment, the valve means may comprise a number of individual valves or similar.
the gas suction means comprise a suction pump comprising a gas inlet fluidically connected to the gas analysis line and a gas outlet fluidically connected to the atmosphere, preferably via an outlet orifice or vent.
the operating means are configured to operate the gas suction means to suck up the gas.
the gas suction means are able to suck up the gas at a given suction flow rate of between 50 and 200 mL/min.
the concentration-measuring means comprise an NO sensor and/or an NO₂ sensor.
the display means comprise a display screen, preferably a touchscreen, that is to say a touch panel.
according to one embodiment, the touchscreen is configured to display an activation key that can be actuated by the user by pressing it, i.e. a touch-actuated key able to instigate an ambient-air analysis procedure.
the touchscreen is configured to display a selection-confirmation key.
the selection-confirmation key is displayed in a window displayed on the screen after the operator has pressed the activation key.
the selection-confirmation key is configured to authorize/begin or to prohibit/cancel the starting of an ambient-air analysis procedure.
starting the ambient-air analysis procedure involves an action by the operating means on the valve means, particularly on the electrically-operated valve, to supply the gas analysis line with ambient air supplied by the secondary sampling line.
the operating means are configured to act on the valve means in response to the user pressing the activation key, preferably after further pressing the selection-confirmation key, in order to place the gas analysis line in fluidic communication with the secondary gas sampling line so as to sample ambient air and subject it to the concentration-measuring means so as to determine the NO and/or the NO₂ content of the sampled ambient air.
according to another embodiment, the operating means are configured to act on the valve means automatically (i.e. without the user pressing or performing any other intervention) at a given activation frequency in order to place the gas analysis line in fluidic communication with the secondary gas sampling line so as to sample ambient air and subject it to the concentration-measuring means so as to determine the NO and/or the NO₂ content of the sampled ambient air, namely so as to periodically measure the NO and/or NO₂ content in the room.
the operating means are configured to act automatically on the valve means, namely to activate the valve means, at an activation frequency of several times per day, preferably once to several times per hour.
the activation frequency is stored in memory, preferably in memory-storage means and/or within the operating means.
the activation frequency is stored in memory in the form of a schedule.
the touchscreen is configured to display the determined NO content and/or NO₂ content of the ambient air.
the operating means are configured to determine the NO and/or NO₂ contents in the ambient air by processing the measurements taken by the concentration-measuring means after a given latency time or delay.
the latency time or delay is comprised between from 10 to 120 seconds, preferably at least 30 sec, typically between 40 and 90 sec.
according to one embodiment, the given latency time or delay begins after the user presses the activation key or preferably after they further press the selection-confirmation key.
according to another embodiment, the latency time begins after actuation of the valve means, i.e. automatic actuation or manual actuation by the user pressing on the touchscreen.
according to one embodiment, the given latency time is predefined and/or stored in memory.

Depending on the embodiment considered, the apparatus of the invention can also comprise one or more of the following additional features:
the operating means are configured to operate the gas suction means, such as a suction pump, to suck up gas coming from the main gas sampling line or from the second gas sampling line.
the main gas sampling line comprises a nonreturn device.
the secondary gas sampling line comprises a filtering means such as an HEPA filter or similar.
the second gas sampling line comprises a calibrated-orifice device; this is preferably positioned between the filtering means and the electrically-operated valve.
the gas analysis line comprises one or more pressure tapping(s) and a calibrated-orifice device.
the given suction flow rate of the suction pump is less than or equal to 400 mL/min, preferably at least 50 mL/min, preferably between 100 and 350 mL/min, more preferably still, between 200 and 300 mL/min.
the gas suction means comprise a suction pump of the type comprising a diaphragm, also known as a diaphragm pump, or of the piezoelectric type, namely one which, in its operation, makes use of the dynamic piezoelectric effect resulting from frequency-based excitation.
the concentration-measuring means comprise NO and/or NO₂ sensors.
the NO/NO₂ measuring means comprise electrochemical sensors.
the concentration-measuring means preferably comprise an oxygen sensor, typically an electrochemical O₂ sensor.
it comprises electrical supply means for supplying electric current to the various elements or components of the apparatus that need this in order to operate, for example to the operating means, to the sensors, to the suction pump, etc. Preferably, the electrical supply means comprise a connection to the mains (110/220 V) and/or a rechargeable battery or other battery, and optionally a current converter.
the operating means are configured to process the measurements coming from the concentration-measuring means and from them deduce values for the NO and/or NO₂ concentration or content in the analysed gas flow.
the measuring means further comprise an O₂ sensor for measuring the O₂ content of the analysed gas flow.
the display means are controlled by the operating means to display the NO and NO₂, and possibly O₂, concentrations, i.e. contents.
the display means are controlled by the operating means to constantly display the NO and NO₂, and possibly O₂, concentrations.
the NO and NO₂ concentrations displayed correspond to those in the surrounding ambient air, i.e. in the room or other location in which the apparatus is situated.
the operating means are configured to process measurements coming from the concentration-measuring means and from them deduce values for the NO and/or NO₂ concentration or content in the analysed gas flow several times per day.
the measuring means are configured to take one or more measurements of the NO and/or NO₂ concentration or content in the ambient air flow between two calibrations of the apparatus.
the measuring means are configured to take one or more measurements of the NO and/or NO₂ concentration or content in the ambient air flow while a patient treatment is ongoing.
the display means are configured to perform one or more acts of displaying on the display screen.
the display screen displays in colour or in black and white, preferably in colour.
the main gas circuit of the NO delivery apparatus comprises flow control means for controlling the flow rate of gas in the main gas circuit carrying the NO/N₂ mixture, preferably at least one electrically-operated valve, notably of proportional type.
the main gas circuit comprises one or more gas passages, such as gas conduits or the like.
the main gas circuit comprises at least one gas inlet through which the NO-containing gas, typically an NO/N₂ mixture, can enter the main circuit of the apparatus.
the gas outlet via which the NO/N₂ mixture leaves the main circuit is connected to the patient circuit by a flexible hose or similar.
the flow control means are controlled by the operating means to allow or prevent any circulation of gas in the main gas circuit; in particular said at least one electrically-operated valve is operated by the operating means (i.e. commanded electrically-operated valve).
the main gas circuit further comprises pressure control means, in particular a gas pressure regulator device.
the operating means comprise at least one microprocessor arranged on at least one electronic board.
said at least one microprocessor implements at least one algorithm.
at least one algorithm is configured to determine the NO and/or NO₂, or even O₂, contents from the measurements taken by the concentration-measuring means of the gas analysis line.
it comprises an external shell or casing.
it comprises memory-storage means, such as a flash memory or similar.
the values for the NO, NO₂ and/or O₂ contents can be stored in memory by the memory-storage means.

Moreover, the invention also relates to an installation for administering gas to a patient, namely dedicated to supplying to a patient, i.e. an individual having need of it, a gaseous mixture containing NO and oxygen, and comprising: the NO delivery apparatus according to the invention; a medical ventilator, such as a resuscitation ventilator; and the patient circuit to which the NO delivery apparatus and the medical ventilator are fluidically connected, and wherein the patient circuit is fluidically coupled to the valve means via the main gas sampling line.

Depending on the embodiment in question, the gas supply installation of the invention can comprise one or more of the following features:
the medical ventilator is a ventilatory assistance apparatus, such as a resuscitation ventilator.
the medical ventilator supplies the patient circuit with a respiratory gas containing oxygen, typically at least 20 vol % approximately of oxygen, preferably at least 20 vol % approximately of oxygen, particularly air or an N₂/O₂ mixture.
the NO delivery apparatus is supplied with an NO-containing gas, in particular an NO/N₂ mixture, by one (or more) gas sources containing gaseous NO.
the one or more gas sources contain an NO/N₂ mixture containing between 100 and 2500 ppm by volume of NO (ppmv), the remainder being nitrogen, preferably less than 1500 ppmv, the remainder being nitrogen, preferably less than 1000 ppmv, the remainder being nitrogen.
preferably, the gas source contains an NO/N₂ mixture containing from 250 to 900 ppm by volume of NO, the remainder being nitrogen, for example of the order of 800 ppm by volume of NO, the remainder being nitrogen.
the one or more gas sources is or are (a) pressurized gas cylinder(s).
the patient circuit comprises an inspiratory branch and an expiratory branch.
the NO delivery apparatus, the medical ventilator and the main gas sampling line of the NO delivery apparatus are fluidically coupled to the inspiratory branch of the patient circuit.
the inspiratory branch and the expiratory branch are connected at a junction piece, such as a Y-piece.
the patient circuit, in particular the inspiratory branch, supplies a respiratory interface, such as a tracheal intubation tube or a breathing mask.
a flow sensor is arranged on the patient circuit, preferably on the inspiratory branch.
the flow sensor is arranged upstream of the site of injection of the gaseous NO, i.e. the NO/N₂ mixture, coming from the NO delivery apparatus.
a gas humidifier is arranged on the inspiratory branch, preferably downstream of the site of injection of the gaseous NO, i.e. of the NO/N₂ mixture, coming from the NO delivery apparatus.
the patient circuit is fluidically connected to an outlet port of the medical ventilator in order to collect and convey the oxygen-containing gas delivered by the medical ventilator, such as air or an N₂/O₂ mixture.
the one or more pressurized gas cylinders contain, when full, an NO/N₂ gas mixture at a pressure of at least 150 to 200 bar abs, or even at least 250 to 300 bar abs.
the patient circuit comprises flexible hoses or conduits.
the flow sensor is electrically connected to the operating means in order to supply these with the flow rate measurements for the gas flow coming from the medical ventilator.
the main gas sampling line of the NO delivery apparatus is fluidically connected to the patient circuit at a gas-sampling site located downstream of the main flow rate sensor, preferably downstream of the gas humidifier.
the gas circuit of the NO delivery apparatus is fluidically coupled to the patient circuit via a gas injection line such as a flexible hose.
the main gas sampling line is fluidically connected to the patient circuit via a sampling conduit, such as a flexible hose.
the sampling conduit is coupled to the patient circuit by means of a connecting piece, such as a T-piece, allowing gas samples to be taken from within the patient circuit, i.e. in the inspiratory branch.
the NO delivery apparatus supplies the patient circuit with an NO/N₂ mixture, so as to dilute the gaseous NO (i.e. the NO/N₂ mixture) in the flow of oxygen-containing respiratory gas, such as air or an O₂/N₂ mixture, and obtain a final gas mixture intended to be administered to the patient.
the final gas mixture comprises essentially nitrogen (N₂), oxygen (O₂) at a concentration of at least 20 vol % approximately, and NO at a content of between 1 and 80 ppmv, typically of the order of 5 to 40 ppmv corresponding to a desired dosage, i.e. an NO/O₂/N₂ gas mixture.
the final gas mixture may contain NO₂ species resulting from the oxidation of some of the NO, preferably less than 2 ppmv of NO₂, more preferably still, less than 1 ppmv of NO₂.

### DEFINITIONS

In general, within the context of the invention:
“ppmv” means parts per million by volume.
“vol %” means percentage by volume.
“NO” denotes nitric oxide.
“NO₂” denotes nitrogen dioxide.
“N₂” denotes nitrogen.
“O₂” denotes oxygen.
the pressures are expressed in bar absolute, abbreviated to “bar”.
the terms “concentration”, “quantity”, “proportion”, “dose” and “content” are considered to be equivalent and interchangeable.
the term “location” is to be interpreted in the broadest sense. It may refer to a room, a ward, a side room, a corridor or other type of room within a hospital establishment (i.e. a hospital, clinic or similar) and also to the passenger space or compartment or an interior space of a transport vehicle, such as an ambulance, a helicopter, an aircraft or the like.
the terms “means of/to/for” are considered to be wholly equivalent to and capable of being substituted by the terms “device of/to/for”, for example the term “operating means” may be replaced by “operating device”, the term “valve means” may be replaced by “valve device”, the “storage means” may be replaced by “storage device”, etc.
a “pressure measurement” means a pressure value (e.g. a numerical value) or a signal representative of such a pressure value, which reflects or corresponds to the gaseous pressure measured by a pressure sensor or the like.
a “measurement of the content of compound A” means a value (e.g. a numerical value) for the compound-A content or a signal representative of such a value which reflects or corresponds to the concentration of compound A in an analysed gas flow.
the terms “upstream” and “downstream” are used in relation to the normal direction of gas flow, namely in the direction leading towards the patient.

### BRIEF DESCRIPTION OF VIEWS OF DRAWINGS

The invention will now be understood more clearly from the following detailed description, provided by way of non-limiting illustration, with reference to the appended figures, in which:

FIG. 1 is a schematic view of one embodiment of an installation for administering gas to a patient and comprising an NO delivery apparatus according to the invention.

FIG. 2 is a schematic depiction of the gas analysis means of an NO delivery apparatus according to the invention, such as that of the installation of FIG. 1.

FIG. 3 to FIG. 5 are examples of displays performed by the NO delivery apparatus according to the invention.

### DETAILED DESCRIPTION

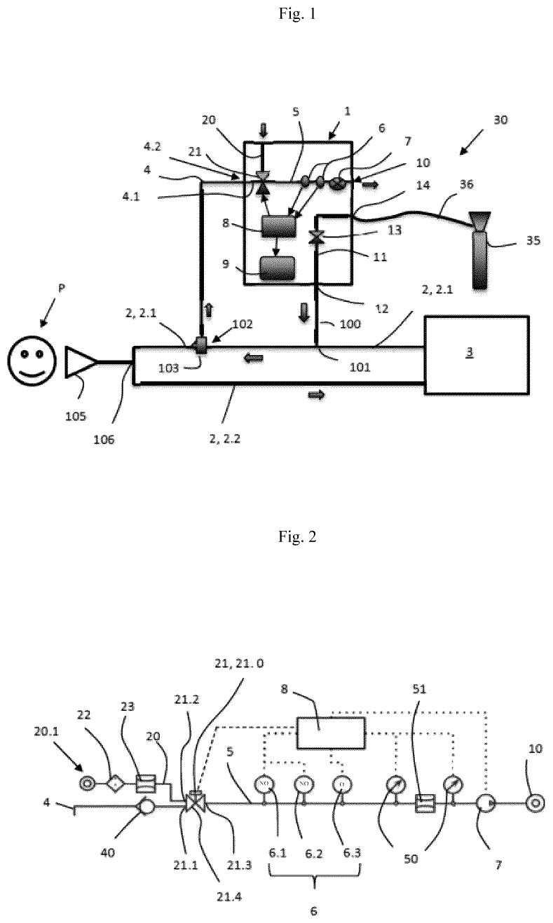

FIG. 1 schematically illustrates one embodiment of a gas delivery installation 30 for supplying an NO-containing gas mixture to a patient having need thereof, comprising an NO delivery device or apparatus 1 for supplying an NO-based gas, typically an NO/N₂ gas mixture, and a medical ventilator 3, that is to say a respiratory assistance apparatus delivering a respiratory gas containing oxygen, typically at least 20% oxygen (O₂) approximately, generally at least 21% oxygen approximately.

Such an installation 30 makes it possible to deliver to a patient P a final gas mixture containing NO at a desired concentration corresponding to a dosage fixed by an anaesthetist or the like, typically between 1 and 80 ppmv of NO (i.e. ppm by volume). The final gas mixture is formed by mixing a flow of NO-based gas, such as an NO/N₂ mixture coming from the NO delivery apparatus 1, and a flow of respiratory gas containing oxygen, typically at least 20% oxygen (O₂) approximately, such as air or an O₂/N₂ mixture, coming from the medical ventilator 3.

The flow of NO-based gas (i.e. NO/N₂) leaving the NO delivery apparatus 1 via an outlet 12 is conveyed by a flexible conduit 100 and is then injected (at 101) into a patient circuit 2, particularly into the inspiratory branch 2.1 of the patient circuit 2, which is also supplied with the oxygen-containing gas flow provided by the medical ventilator 3. The patient circuit 2 comprises for example flexible hoses, for example made of polymer.

The flow rate of the flow of O₂-based gas flowing in the patient circuit 2 is measured by a main flow rate sensor (not shown) arranged on the patient circuit 2, i.e. on the inspiratory branch 2.1, which sensor is electrically connected to the operating means 8 of the NO delivery apparatus 1 so as to supply them with flow rate signals or measurements reflecting the flow rate of the flow of oxygen-containing gas supplied by the medical ventilator 3 circulating in the patient circuit 2 towards the patient P. The main flow rate sensor can be a flow rate sensor of the mass flowmeter type or pressure differential type, or similar.

The final gas mixture obtained by mixing the flows of NO/N₂ and of air or of an O₂/N₂ mixture is formed essentially of oxygen and nitrogen, and of NO at the desired dosage, typically between 1 and 80 ppmv of NO; it may contain unavoidable impurities, for example argon or the like. It is administered to the patient P, during their inspiratory phases, by means of a respiratory interface 105, for example a breathing mask, a tracheal intubation tube or any other suitable interface.

The gases exhaled by the patient during their expiratory phases are collected by an expiratory branch 2.2 of the patient circuit 2. The inspiratory and expiratory branches 2.1, 2.2 comprise conduits, pipes, hoses, passages, tubes or similar, for example flexible polymer hoses, that are able and configured to convey the gas flows, and which are fluidically coupled to a connecting piece 106, such as a Y-piece or similar, which is also connected to the respiratory interface 105. The respiratory interface 105 makes it possible to deliver the gas to the patient P, in particular during the inspiratory phases, and to collect the (CO₂-rich) gases exhaled by the patient P, in particular during the expiratory phases.

During the operation of the installation 30, the gas flow circulating in the inspiratory branch 2.1 of the patient circuit 2, that is to say going from the mechanical ventilator 3 to the patient P, is inhaled by the patient, while the gases exhaled by same, i.e. gases enriched in CO₂, are conveyed via the expiratory branch 2.1 to the ventilator 3, where they are evacuated to the atmosphere via a venting orifice (not shown) or the like.

In general, the NO delivery apparatus 1 is of conventional architecture and operation as regards the supply of NO, typically an NO/N₂ mixture. It comprises (not shown) an internal main gas circuit 11 for conveying the NO-containing gas, in particular one or more internal conveying lines for gas, typically an NO/N₂ mixture comprising from 100 to 2000 ppmv of NO (remainder N₂), typically less than 1000 ppmv.

Means 13 for controlling the flow rate, such as an electrically-operated valve, and/or for controlling the pressure of the gas flow, such as a gas pressure regulator device, are arranged on the main gas circuit 11. The flow rate control means 13 may be operated by the operating means 8 of the NO delivery apparatus 1, notably on the basis of flow rate signals or measurements supplied by the main flow rate sensor.

The operating means 8 comprise one or more (micro)processor(s) carried by one or more electronic boards and implementing one or more algorithms configured to process the measurements or other information, data or similar.

The main flow rate sensor is advantageously arranged on the inspiratory branch 2.1, upstream of the injection site 101 where NO (i.e. the NO/nitrogen mixture) is injected into the inspiratory branch 2.1 and mixes with the oxygen-containing gas flow coming from the medical ventilator 3.

The NO/N₂ mixture passing through the NO delivery apparatus 1 comes from one or more gas sources 35, typically one or more cylinders of compressed gas (not shown) containing the NO/N₂ gas mixture at a pressure of up to 150 bar, or even 180 to 200 bar, or more. The gas source(s) 35 is (are) fluidically coupled via one or more flexible conduit(s) 36 to (at least) a gas inlet 14 of the main gas circuit 11 of the NO delivery device 1.

As has already been explained, the NO delivery apparatus 1 allows the NO-based (i.e. NO/nitrogen) mixture to be supplied/injected (at 101) into the patient circuit 2 so that it becomes mixed (i.e. diluted) therein with the flow of oxygen-containing gas coming from the medical ventilator 2, for example an N₂/O₂ mixture or air.

The final NO/O₂/N₂ gas mixture that is then administered to the patient P via the respiratory interface 105, contains predominantly nitrogen (N₂), oxygen (O₂) at a content of approximately at least 20% vol., and NO at a content of between 1 and 80 ppmv, typically of the order of 5 to 40 ppmv corresponding to the desired dosage, which is notably dependent on the population being treated, i.e. newborns, children, adolescents or adults and on the condition being treated.

Indeed, nitric oxide or NO, when inhaled, dilates the pulmonary vessels and increases oxygenation by improving gas exchange. These properties are used to treat various medical conditions such as persistent pulmonary hypertension of the newborn (PPHN), acute respiratory distress syndrome (ARDS) observed mainly in adults, or else pulmonary hypertension (PH) in heart surgery, observed in adults or children, as described in particular by EP-A-560928, EP-A-1516639 and U.S. Pat. No. 10,201,564.

However, on account of the presence of O₂ in the final NO/O₂/N₂ gas mixture, some of the NO present therein will oxidize and form toxic NO₂ species.

It is therefore essential to ensure that the concentration of NO in the final NO/O₂/N₂ gas mixture does indeed correspond to the dosage desired by the physician and that, furthermore, the quantity of toxic NO₂ species is limited, typically less than a few ppmv, in general less than 0.5 ppmv.

To do this, the NO delivery apparatus 1 of FIG. 1 incorporates gas analysis means 5, 6, i.e. a gas analyser, which are arranged in the external shell or casing, for example made of polymer, of the NO delivery apparatus 1 and which make it possible to monitor, i.e. measure, the concentrations of NO and NO₂ in the final gas mixture, as explained below.

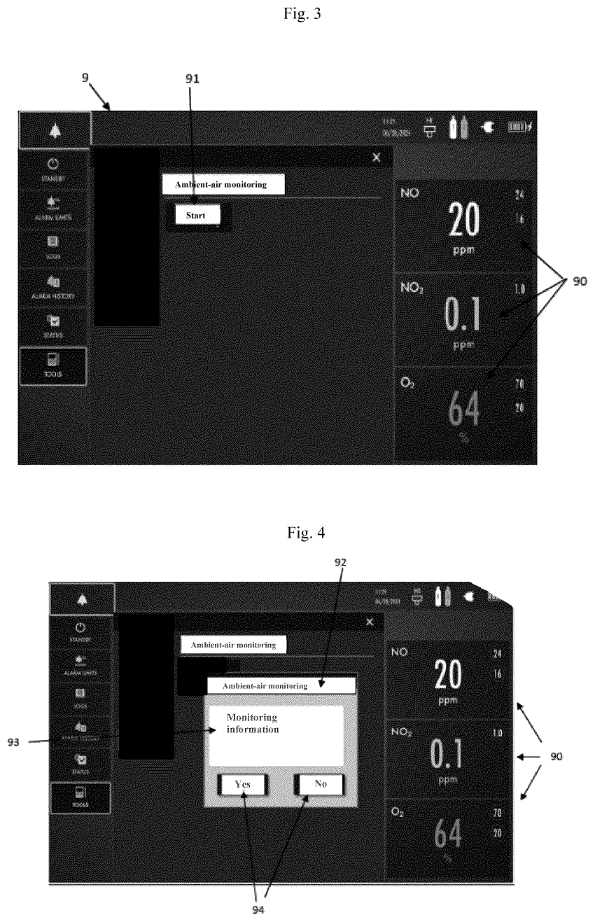

As detailed in FIG. 2, the gas analysis means 5, 6 comprise a gas analysis line 5 comprising concentration-measuring means 6 configured to perform NO and/or NO₂, or even oxygen, content measurements within the gaseous flow present in said gas analysis line 5.

A main gas sampling line 4, such as a flexible hose, fluidically couples the patient circuit 2 to the gas analysis line 5 so that gas samples can be taken from the circuit 2, i.e. from the inspiratory branch 2.1, and conveyed to the gas analysis line 5 so that they can be brought into contact with the concentration-measuring means 6 so that these can take measurements of the NO and NO₂ and/or O₂ contents.

As a preference, the concentration-measuring means 6 comprise NO, NO₂ and possibly O₂ sensors, for example of electrochemical or some other type. The sensors may each be dedicated to one given compound (NO, NO₂ or O₂) or may be combined to measure the contents of several compounds, particularly NO and NO₂.

The concentration measurements taken by the concentration-measuring means 6 are transmitted to the operating means 8 which process them and from them deduce values for the NO and NO₂ and/or O₂ contents.

In order to allow gas to be sampled, namely to allow gas samples to be taken, from the circuit 2, gas suction means 7, such as a suction pump, are arranged downstream of the main gas sampling line 4, typically in the gas analysis line 5, so as to suck up these gas samples. The suction pump may be of the diaphragm pump or piezoelectric pump type.

As illustrated in FIG. 2, the gas analysis line 5 may also comprise pressure tappings or sensors 50 arranged on each side of a calibrated-orifice device 51, notably so as to constitute a differential-pressure sensor, these being electrically connected to the operating means 8 which can be configured so that a gas flow rate can be determined from differential pressure measurements.

Moreover, the NO delivery apparatus 1 also comprises display means able to display various pieces of information, values, data, alarms, graphics, icons, windows, curves, gauges or similar. As a preference, these means comprise a display screen 9, preferably a touchscreen, that is to say a touch panel. The displays are preferably displayed in colour.

In particular, the touchscreen 9 is also able to display one or more touch-actuated virtual keys for notably performing one or more activation, selection or validation operations, for example in selection menus or in virtual windows likewise displayed on the screen 9. One or more such virtual keys can be actuated by the user by pressing on the window/panel-tile of the screen 9, i.e. can be activated or selected by the user, i.e. the caregiver, touching or pressing it with their finger or other digit.

Thus, the touchscreen 9 is also able to display the NO and/or NO₂, and possibly O₂, contents determined by the operating means 8 from the measurements taken by the concentration-measuring means 6, typically by the NO, NO₂ and possibly O₂ sensors. This display enables the caregivers to ensure that the final gas e.g. (NO/N₂/O₂) mixture contains the desired NO dosage, a non-hypoxic oxygen content (i.e. >20% vol.) and, conversely, does not contain an excessive quantity of toxic NO₂ species.

The operating means 8, particularly the microprocessor(s), are also configured to control all or some of the displays displayed on the display screen 9, i.e. on the display means, particularly the determined values for the NO and NO₂ contents.

The electrical power for the NO delivery apparatus 1, in particular for the operating means 8, the sensors, the pump 7, the screen 9, etc. is provided by an electrical current source and/or electrical supply means (not shown), for example a connection to the mains (110/220 V) current, such as an electrical cord and connection socket, and/or one or more electric, preferably rechargeable, batteries, and/or a current transformer.

The apparatus 1 may also comprise memory-storage means, such as a flash memory or similar. This may be incorporated into the operating means 8, for example carried by an electronic board.

In the embodiment of FIG. 1, the main gas sampling line 4, such as a flexible hose, is coupled, by its downstream end, to the gas analysis line 5 via a connecting section 4.1 housed within the apparatus 1. The connecting section 4.1 comprises an inlet orifice 4.2 to which the main gas sampling line 4 is coupled; for example, the inlet orifice 4.2 may be borne by a fluidic connector or coupling or similar. According to another embodiment (not shown), the main gas sampling line 4 may be coupled directly to the gas analysis line 5.

The upstream end of the main gas sampling line 4 is coupled (at 102) to the patient circuit 2 via a suitable coupling device 103, for example a T-piece fluidic connector or similar, arranged downstream of the point 101 of injection of NO into the patient circuit.

The gas suction means 7, typically a suction pump, allow the gas to be circulated in the main gas sampling line 4 and in the gas analysis line 5, via the section 4.1, by creating a depression that gives rise to a flow or stream of gas that sweeps over the NO and NO₂ (or even also O₂) concentration-measuring means 6, which is to say the sensors of the gas analysis line 5, so that these can take the desired concentration measurements. The suction flow rate is preferably comprised between 50 and 350 mL/min, for example of the order of 200 to 250 mL/min.

The gas flow to be analysed is therefore sucked in by the pump 7 and circulates through the gas analysis line 5 so as to be brought into contact with the NO₂ and NO sensors, which will then measure the NO₂ and NO concentration of the gas flow and supply these measurements to the operating means 8, which will then return them to the user, such as a physician or another caregiver, by ordering them to be displayed on the touchscreen 9, which may form part of a graphical user interface (GUI) of the NO delivery apparatus 1.

The gas analysis line 110 may also comprise one or more other sensors, particularly an oxygen sensor, and the oxygen concentration may also be displayed on the screen 9 so as to allow the user to monitor it and assure themself that the gas mixture supplied to the patient is not hypoxic.

Of course, if necessary, the operating means 8 can also process the NO and NO₂ measurements before ordering that they be displayed on the screen 9, in particular in order to perform compensation of these values so as to take account of environmental factors which can influence the measurements, such as atmospheric pressure, temperature and/or humidity, and to which the gas mixture may have been subjected.

Advantageously, the operating means 8 comprise one or more electronic control board(s) and a control unit involving one or more (micro)processor(s), typically a microcontroller or the like; preferably the electronic control board incorporates the control unit. Preferably, the operating means 8 also control, i.e. operate, the suction pump 7 so that the flow rate circulating in the gas analysis line 5 is kept constant and equal to the desired target flow rate, e.g. comprised between 50 and 350 mL/min.

Following analysis, the gas flow is discharged into the atmosphere after having passed through the gas analysis line 5 and through the suction pump 7 situated between the concentration-measuring means 6, i.e. the NO and NO₂ (or even also O₂) sensors, and an outlet orifice or vent 10 in fluidic communication with the ambient air, i.e. the atmosphere of the room in which the apparatus 1 is situated.

The ambient air is therefore “polluted” with these compounds NO and NO₂.

Now, certain caregivers, chiefly allied health professionals, are afraid of being exposed to NO and to NO₂ when performing care duties in the room in which patients are being treated by the administration of NO, for example in a ward or a resuscitation room, causing them anxiety and moreover potentially affecting their health.

Therefore, in an attempt to overcome this, according to the invention, the NO delivery apparatus has been improved to make it possible to determine and display the concentration of the compounds NO and/or NO₂ in the ambient air of the hospital room in which the NO supply installation 30 is situated and/or used, for example a ward, a resuscitation room or similar.

Specifically, knowledge of their level of exposure to the compounds NO and NO₂ present in the ambient air allows caregivers to reduce their level of anxiety and moreover protect their health by allowing them to ensure that they are not being subjected to excessive doses, typically exceeding the exposure limits.

In order to do this, according to the invention and as visible in FIG. 1 and detailed in FIG. 2, there have been provided in the NO delivery apparatus 1, a secondary gas sampling line 20 fluidically communicating with the ambient atmosphere through an ambient-air inlet 20.1, and valve means 21 fluidically coupled to the main gas sampling line 4, to the secondary gas sampling line 20 and to the gas analysis line 5.

The main gas sampling line 4 here comprises a nonreturn valve 40 arranged upstream of the valve means 21 so as to prevent any backflow of gas towards the patient circuit 2.

The valve means 21 comprise a commanded electrically-operated valve, namely one that is operated by the operating means 8, for example a 3-way electrically-operated valve. It typically comprises a valve body 21.4 with internal gas passages (not visible), comprising: a main valve inlet 21.1 fluidically coupled to the main gas sampling line 4; a secondary valve inlet 21.2 fluidically coupled to the secondary gas sampling line 20; and a valve outlet 21.3 fluidically coupled to the gas analysis line 5.

Moreover, as illustrated in FIG. 2, it may be seen that the secondary gas sampling line 20 preferably comprises:
a filtering means 22, such as an HEPA filter or similar, situated downstream of the air inlet 20.1 and serving to purify the air by removing any dust, spores, bacteria or other aerosols that it might contain; and
a calibrated-orifice device 23, arranged between the filtering means 22 and the electrically-operated valve 21.0 and able to generate (where necessary) a drop in pressure of the airflow in the secondary line 20 that is equivalent to the drop that occurs in the main line 4, for example when the latter is fitted for example with a “water trap” type device (not shown). Specifically, such “equalizing” enables the pump 7 to remain at the same pump speed when the valve means 21, typically the electrically-operated valve 21.0, are switched over.

More specifically, the operating means 8 are configured to control both the valve means 21, i.e. the electrically-operated valve 21.0, for placing the gas analysis line 5 in fluidic communication with one, or alternatively the other, of the main and secondary gas sampling lines 4, 20, namely one of them or the other one, but never both at the same time.

In order to do this, the operating means 8 are electrically connected to said valve means 21 so as to transmit to them command signals aimed at causing the supply of gas to the electrically-operated valve 21.0 to be switched towards one or the other of the main and secondary gas sampling lines 4, 20 according to whether the desire is to determine the NO and/or NO₂ composition of the final gas flow coming from the patient circuit 2 or of the ambient air surrounding the apparatus 1.

More specifically, the valve means 21, namely the electrically-operated valve 21.0, are commanded by the operating means 8 so as to fluidically connect the gas analysis line 5 to the main gas sampling line 4. At that moment, the gas coming from the patient circuit 2 may enter the body of the electrically-operated valve 21.4 via the main valve inlet 21.1 and then pass through the body of the electrically-operated valve 21.4 via the internal passages thereof before reemerging via the valve outlet 21.3 that is fluidically coupled to the gas analysis line 5 so that it can be analysed by the concentration-measuring means 6 in order to measure the NO and NO₂ content, as explained hereinabove, or even the O₂ content, namely by using the NO sensor 6.1, the NO₂ sensor 6.2 and the O₂ sensor 6.3 of the gas analysis line 5.

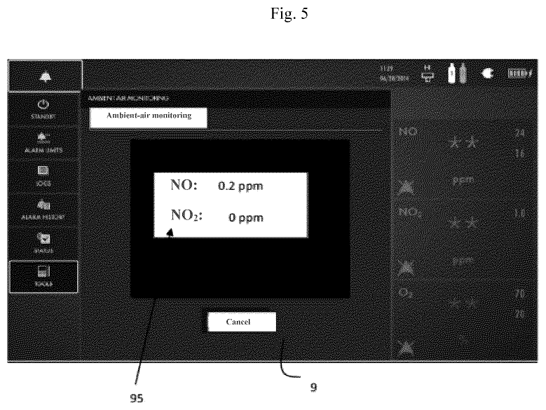

Once the measurements have been taken by the NO sensor 6.1, the NO₂ sensor 6.2 and the O₂ sensor 6.3 and transmitted to the operating means 8, they are processed by the latter which also control their displaying (at 90) on the screen 9 while the treatment of the patient P is ongoing, as illustrated in FIG. 3 and FIG. 4, enabling the caregivers to assure themselves that the final composition of the gas supplied to the patient does indeed conform to the expected dosage and does not contain toxic NO₂ species exceeding the maximum permissible limit(s).

This corresponds to normal and conventional operation of the NO delivery apparatus 1.

When a caregiver wishes to know their own level of exposure to the compounds NO and/or NO₂ present in the ambient air, namely in the hospital environment, passenger space or similar in which the installation 30 and the apparatus 1 are situated, so as to assure themselves that they are not being subjected to excessive doses of these compounds, according to one embodiment of the invention that caregiver may press the touchscreen 9 of the NO delivery apparatus 1, particularly a dedicated activation key 91, for example a key entitled “Start”, as illustrated in FIG. 3.

Optionally, as illustrated in FIG. 4, the pressing of the activation key 91 may open a window 92 on the display screen 9 inside which window information relating to the monitoring of the ambient air is displayed (at 93), together with two selection-confirmation keys 94, i.e. a “yes” key and a “no” key or similar, so that the starting of the ambient-air analysis procedure can be initiated or cancelled.

In all cases, the act (or acts) of pressing, i.e. finger contact with or pressure on, the touch panel of the screen 9, namely the activation key 91 and optionally one or the other of the selection-confirmation keys 94, will then be detected by the operating means 8, for example by means of an activation signal transmitted by the touch panel of the screen 9 to said operating means 8, which will then act on the valve means 21 in response to the user pressing the activation key 91 (and possibly then the selection-validation key 94, i.e. the “yes” key), so as to place the gas analysis line 5 in fluidic communication with the secondary gas sampling line 20 so as to allow the ambient air to be sampled and subjected, preferably after filtration (at 22), to the concentration-measuring means 6, i.e. in particular to the NO sensor 6.1 and the NO₂ sensor 6.2, so that the NO and NO₂ concentrations in the sampled ambient air can be measured or determined.

According to another embodiment, the operating means 8 may be configured to act on the valve means 21 automatically at a given activation frequency in order to place the gas analysis line 5 in fluidic communication with the secondary gas sampling line 20 so as to sample ambient air and subject it to the concentration-measuring means so as to determine the NO and/or the NO₂ content of the sampled ambient air, as already stated.

In this embodiment, the delivery apparatus 1 therefore takes periodic measurements of the NO and/or NO₂ contents in the room by automatically acting on the valve means 21 to activate them at a given activation frequency, typically several times per day, preferably once to several times per hour.

The activation frequency is stored in memory, for example in the form of a daily schedule or similar, preferably in memory-storage means and/or directly within the operating means 8, for example inside a dedicated algorithm.

In other words, in response to pressure on the activation key 91 (and possibly on the validation key), or according to the defined activation frequency, the electrically-operated valve 21.0 switches over to halt the passage of final gas coming from the patient circuit 2 through the body of the electrically-operated valve 21.4 via the main valve inlet 21.1 but rather allow ambient air carried by the secondary gas sampling line 20 to pass through the body of the electrically-operated valve 21.4 via the secondary valve inlet 21.2. The flow of ambient air, that is sucked in by the suction means 7, as already explained, is first of all filtered by the filtering means 22 and can then pass through the body of the electrically-operated valve 21.4 via internal passages in order to re-emerge therefrom via the valve outlet 21.3 that is fluidically coupled to the gas analysis line 5 thus allowing the composition of the air to be analysed.

As before, once the measurements have been taken by the NO sensor 6.1 and NO₂ sensor 6.2, these measurements are transmitted to the operating means 8 which process them and also control their displaying (at 95) on the screen 9, as illustrated in FIG. 5, so as to allow the caregivers to know the composition of the ambient air in terms of the compounds NO and NO₂.

In this case, the values for the NO, NO₂ and O₂ concentrations previously measured in the final gas flow coming from the patient circuit are no longer displayed (at 90), unlike in FIG. 3 and FIG. 4.

In general, the NO and NO₂ values measured in the ambient air can be displayed (at 95) in almost real-time, in response to a choice made by/pressure applied by the user or, depending on the embodiment, automatically at the activation frequency stored in memory.

However, their display 95 preferably comes after a slight delay, namely after a latency time ranging from 10 to 60 seconds, preferably of at least 30 sec, for example of the order of 40 to 45 sec, following activation of the valve means 21 by the operating means 8, so as to allow the values measured by the NO and NO₂ sensors 6.1, 6.2 to “stabilize”, namely so as to limit measurement fluctuations caused by a residual pressure of final gas coming from the patient circuit 2 and liable to mix with the filtered atmospheric air flow entering the analysis line 5.

Now, this mixing between these two flows at the start of measurement, as a result of the unavoidable dilution of part of the final gas (i.e. NO/O₂/N₂) in the analysis line 5 by the filtered air flow entering the analysis line, after the electrically-operated valve 21.0 of the valve means 21 has been switched over, may give rise to small fluctuations in the composition of the gas and therefore slightly falsify the measurements.

It is therefore recommended to wait until the analysis line 5 contains only filtered ambient air, namely until the final gas has been discharged to the atmosphere via the pump 7 and the outlet orifice or vent 10.

In general, it is considered that after 1 to 2 minutes, the measurements taken by the NO and NO₂ sensors 6.1, 6.2 can be considered to have “stabilized” and to properly reflect the NO and NO₂ contents present in the ambient air of the room. These values may also be logged by the memory-storage means or any other means of saving them.

Generally, the gas administration installation 30 comprising the NO delivery apparatus 1 according to the invention is suitable for use in treating various pulmonary pathologies, such as persistent pulmonary hypertension of the newborn (PPHN), acute respiratory distress syndrome (ARDS) and/or pulmonary hypertension (PH) in heart surgery, namely in different patient populations, particularly neonates, adults, adolescents or children.

What is claimed is:

1. NO delivery apparatus (1) comprising:
a main gas circuit (11) for carrying an NO-containing gas as far as at least one gas outlet (12) configured to be fluidically coupled to a patient circuit (2) carrying an oxygen-containing respiratory gas,
a main gas sampling line (4) configured to be fluidically coupled to said patient circuit (2),
a gas analysis line (5) comprising concentration-measuring means (6) configured to perform NO and/or NO₂ content measurements within the gaseous flow present in said gas analysis line (5),
gas suction means (7) configured to suck the gas into at least the gas analysis line (5) and cause it to circulate therein,
microprocessor-containing operating means (8) configured to process the concentration measurements taken by the concentration-measuring means (6) and from them deduce at least an NO and/or an NO₂ content, and
display means (9) configured to display said at least one NO and/or NO₂ content,
characterized in that it further comprises:
a secondary gas sampling line (20) fluidically communicating with the ambient atmosphere, and
valve means (21) fluidically coupled to the main gas sampling line (4), to the secondary gas sampling line (20) and to the gas analysis line (5), the operating means (8) being further configured to control the valve means (21) in order to place the gas analysis line (5) in fluidic communication with one or, alternatively, with the other, of the main gas sampling line (4) and secondary gas sampling line (20).

2. Apparatus according to claim 1, characterized in that the valve means (21) comprise an electrically-operated valve (21.0) comprising:
a main valve inlet (21.1) fluidically coupled to the main gas sampling line (4),
a secondary valve inlet (21.2) fluidically coupled to the secondary gas sampling line (20), and
a valve outlet (21.3) fluidically coupled to the gas analysis line (5).

3. Apparatus according to claim 1, characterized in that the operating means (8) are configured to control the valve means (21), particularly the electrically-operated valve (21.0), in order to place the gas analysis line (5) in fluidic communication with:
either the main gas sampling line (4) in order to supply the gas analysis line (5) with gas coming from the patient circuit (2),
or the secondary gas sampling line (20) in order to supply the gas analysis line (5) with ambient air coming from the atmosphere, i.e. the air in the room and surrounding the apparatus.

4. Apparatus according to claim 1, characterized in that the gas suction means (9) comprise a suction pump comprising a gas inlet fluidically connected to the gas analysis line (5) and a gas outlet fluidically connected to the atmosphere, preferably via an outlet orifice (10).

5. Apparatus according to claim 1, characterized in that the operating means (8) are configured to operate the gas suction means (7) to suck up the gas, preferably at a given suction flow rate of between 50 and 350 mL/min.

6. Apparatus according to claim 1, characterized in that the concentration-measuring means (6) comprise an NO sensor (6.1) and/or an NO₂ sensor (6.2).

7. Apparatus according to claim 1, characterized in that the display means comprise a display touchscreen (9).

8. Apparatus according to claim 4, characterized in that:
the touchscreen (9) is configured to display an activation key (91) and possibly a selection-confirmation key (94) that the user can actuate by pressing it, and
the operating means (8) are configured to act on the valve means (21) in response to the user pressing the activation key (91) and possibly the selection-confirmation key (94) in order to place the gas analysis line (5) in fluidic communication with the secondary gas sampling line (20) so as to sample ambient air and subject it to the concentration-measuring means (6) so as to determine the NO and/or the NO₂ content of the sampled ambient air.

9. Apparatus according to claim 1, characterized in that the operating means (8) are configured to act on the valve means (21) automatically at a given activation frequency in order to place the gas analysis line (5) in fluidic communication with the secondary gas sampling line (20) so as to sample ambient air and subject it to the concentration-measuring means (6) so as to determine the NO and/or the NO₂ content of the sampled ambient air.

10. Apparatus according to claim 9, characterized in that the activation frequency stored in memory is several times per day, preferably once to several times per hour.

11. Apparatus according to claim 1, characterized in that the touchscreen (9) is configured to display (95) the determined NO content and/or NO₂ content of the ambient air.

12. Apparatus according to claim 1, characterized in that the operating means (8) are configured to determine the NO and/or NO₂ contents in the ambient air by processing the measurements taken by the concentration-measuring means (6) after a given latency time.

13. Apparatus according to claim 12, characterized in that the latency time is comprised between from 10 to 120 seconds.

14. Apparatus according to claim 10, characterized in that the activation frequency is stored in memory in the form of a daily schedule.

15. Installation (100) for administering gas to a patient (P), comprising: the NO delivery apparatus (1) according to claim 1; a medical ventilator (3); and the patient circuit (2) to which the NO delivery apparatus (1) and the medical ventilator (3) are fluidically connected, and wherein the patient circuit (2) is fluidically coupled to the valve means (20) via the main gas sampling line (4).
